(19) United States
(12) Patent Application Publication
(10) Pub. No.: US 2020/0199515 A1
(43) Pub. Date: Jun. 25, 2020

(54) Flexible device and its application for bio-cell in-vitro electrical and mechanical stimulation and characterization

(71) Applicant: Erin Glenn Roberts, Fogelsville, PA (US)

(72) Inventor: Erin Glenn Roberts, Fogelsville, PA (US)

(21) Appl. No.: 16/370,895

(22) Filed: Mar. 30, 2019

Related U.S. Application Data
(60) Provisional application No. 62/625,049, filed on Feb. 1, 2018.

(51) Int. Cl. C12M 1/42; C12N 5/077; A61F 2/00

(52) U.S. Cl. CPC C12M 35/02; C12M 35/04; A61F 2002/0086; A61F 2/0077; C12N 5/0657

(57) ABSTRACT

Disclosed below is a device comprising a base 112, polymer walls 105, comb fingers 104, groove/ridge architecture 107, metal film 108, comb buses 103, electrical ground electrode 109, and polymer well 101 for electrical and mechanical stimulation as well as for measuring of contractile force and conduction velocity of the cellular sheet/cluster 102 in response to mechanical and electrical stimulation of the cell source as it grows into a multilayer cellular sheet on the device. This allows cell sheets to be cultured and conditioned to be compatible with the patient's cardiac environment in vitro, prior to sheet release and implantation. Major innovative elements of this device include: real-time data for rich understanding of engineered tissues as they are grown, ability to expose engineered tissues to patient-derived stimuli (specifically localized electrical stimuli, mechanical stimuli, and micro-architecture), and the option to implant after characterization.

### CROSS REFERENCE TO RELATED APPLICATIONS

This application is a continuation of U.S. provisional patent application No. 62/625,049, filed Feb. 1, 2018.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

Cardiovascular diseases (CVDs) are the most common cause of death worldwide with an estimated 17.7 million people dying from them in 2015, 31% of all global deaths. Ischaemic heart disease and stroke are the primary conditions responsible for these deaths and have been so for the past 15 years.

Currently the most widely used surgical patch solutions for repairing damaged myocardium include synthetic materials such as Dacron, Gore-Tex, and ePTFE as well as biological materials such as autologous, allogenic, and xenogenic pericardium. However, these patches are unable to grow and electromechanically incorporate upon implantation within the resident heart tissue, often resulting in patch failure and the need for re-operation.

Tissue engineered patches fabricated from stacked cellular sheets provide one solution to this problem. Cell sheets can be fabricated from many cell sources (e.g., induced pluripotent stem cell derived cardiomyocytes, mesenchymal stem cells) by seeding the cells onto a substrate (e.g., PNIPAAM-coated PDMS, alginate hydrogel) that allows for proliferation, extracellular matrix deposition, and triggered release of the cell sheet from the substrate. Single layer cell sheets can be stacked to provide a scaffold-less patch implant that is made from autologous, functional tissue and better incorporates at the site of implantation.

Native myocardium is formed from a high density of aligned myocytes and supporting cells that contract synchronously according to electrical signal propagation throughout the tissue. In order to satisfy these structural and functional needs (conduction velocity of 25 cm/s, contractility of 20-50 mN/mm², myocardial stiffness of 20-500 kPa, see for instance, M. Radisic and K. L. Christman, Mayo Clin Proc, 2013, 88, 884-898) with a tissue-engineered construct, an abundant and compatible cell population is needed to ensure reliable electromechanical function once implanted.

Cardiomyocyte renewal in postnatal mammals is on the order of 1% per year and declines with age. The number of autologous cardiomyocytes that can be isolated from discarded heart tissue is insufficient to allow for the construction of effectively sized cell sheets. Therefore, cardiomyocytes are produced in vitro from various stem cell sources, but these are usually of an immature phenotype, not able to meet the tissue requirements of the heart. Implanting immature myocyte cells into a mature cardiac environment can result in electromechanical mismatch and arrhythmia.

It is important to understand how stem cell derived-cardiomyocyte cell sheet patches will respond to a cardiac environment and how to improve their compatibility. Immature cardiomyocytes produced in vitro have been shown to respond positively (increased maturity) to electrical and mechanical conditioning. A device that electromechanically evaluates myocardial patches intended for patient implantation while simultaneously conditioning them into a more mature phenotype could result in production of safer, more cardiac-compatible tissue-engineered solutions to current CVD problems. The device presented here was designed to satisfy these two main objectives. The bio-MEMS device described herein has been designed to provide electrical and mechanical stimulation and also measures contractile force and conduction velocity simultaneously as cell sheets are fabricated and mature on the device.

Multiple electrical and mechanical methods for cardiomyocyte characterization and conditioning already exist in the field. Well known technologies for cardiac characterization include micro-pillar arrays, which measure individual cellular contractility by tracking the displacements of polymeric micro-pillars as cells sitting on top bend the structures (N. E. Oyunbaatar, D. H. Lee, S. J. Patil, E. S. Kim and D. W. Lee, Sensors (Basel), 2016, 16, 1258) and microelectrode arrays, which stimulate cells and record their responses allowing for the analysis of action potentials and conduction velocities (T. Trantidou, C. M. Terracciano, D. Kontziampasi, E. J. Humphrey, T. Prodromakis, Sci Rep, 2015, 5, 11067).

There are also several existing techniques that combine both mechanical and electrical stimulation as recently reviewed by Stoppel et al in Adv Drug Deliver Rev, 2016, 96, 135-155. Most electrical stimulation setups use two electrodes to provide bulk field stimulation to the culture container, directing electrical impulses through the culture media. Mechanical stimulation has been performed in 2D and 3D with the following three generic methods: varying substrate stiffness, stretching a substrate upon which cells are attached over time, and dynamic stretching and relaxing of such a substrate. Multiple research groups have shown that mechanical and electrical conditioning of cellular constructs can result in tissue remodeling, cellular alignment, increased cardiac protein expression, and improved compatibility and incorporation into the body upon implantation. These techniques and their associated designs do not allow for the level of specificity in measuring mechanical and electrical conductivity tissue properties achievable by this invention.

In addition, the following related, more specific prior art has been identified. In patent application US 2013/0137132 A1, ‘Cardiomyocyte Containing Device, Manufacturing Method and Measuring Method,’ a device for in vitro cardiac electrophysiology screening is disclosed. This device allows for the seeding of cells onto a flexible substrate that can be stretched and is patterned with grooves and multi-electrode structures. The multi-electrode structure allows for more specific stimulation and electrical conductivity measurement than those reviewed by Stoppel et al, however, US 2013/0137132 A1 does not have the following capabilities which this invention does: mechanical activity of the cells on the device cannot be measured, not all cells in the culture are impacted by the groove and electrode patterning, and there is not a mechanism in place for cell release from the device.

The Chinese patent application CN108467835A, ‘Micro-fluidic chip for cardiac-muscle-cell three-dimensional function culturing and preparing method and mechanical-electrical-characteristic detection method,’ aims to evaluate and condition tissue strips. However it does not have cell sheet capabilities cell sheets being single layered or otherwise much thinner constructs that allow for more specific measurements across the sheet. This limits the ability of the device to track cell duster properties as they mature and form full sheets.

The Biowire system is a cell laden collagen gel which compacts around a suture, forming a wire like structure that can be electrically stimulated by electrodes in the media solution; described in L. Lu, M. Mende, X. Yang, H. F. Korber, H. J. Schnittler, S. Weinert, J. Heubach, C. Werner and U. Ravens, Tissue Eng Part A, 2013, 19, 403-414. In certain iterations of the device, beat induced deflections of plastic wires on either end of the tissue are used to measure total force. This and similar systems such as disclosed in the patent US20140220555A1, ‘In vitro microphysiological system for high throughput 3d tissue organization and biological function,’ where deflection of a polymer microcantilever is used to readout force information are often coupled with in media electrodes that stimulate the entire cell culture. The design and resulting capability for electrical stimulation and mechanical measurement is at the tissue level, rather than the cell level, limiting the specificity of measurement/readout.

Specific platforms that combine electrical and mechanical stimulation include the work by Morgan et al (J Tissue Eng Regen M, 2017, 11, 342-353) where the bioreactor uses distension of a latex tube to stretch a circular fibrin and cell construct, carbon rod electrodes inserted in the media can apply electrical pulses, and the two types of stimulation can be offset by a specified amount. Recent related work also includes that of B. Wang et al, Langmuir, 2013, 29, 11109-11117 and Miklas et al Biofabrication, 2014, 6, 024113 where systems combine stimulating electrodes in the culture media with methods to either introduce static or pulsing stretch. Again, these platforms have not been designed to couple co-stimulation with measurement of electromechanical properties at the cellular-level.

The previously described devices and techniques can measure electrical and mechanical properties of an entire piece of tissue as well as condition these tissues, but provide no greater level of detail on a cellular level. Being able to understand the cell-level properties of a tissue and target these during both electrical and mechanical conditioning is valuable, particularly when such information can be gathered over the period of time when individual cells form the sheet or tissue. This is a capability beyond those of the devices described above and a key differentiator of the following device design.

### BRIEF SUMMARY OF THE INVENTION

This invention relates to devices for stimulating, testing, and recording signals from biological cells including cardiomyocyte cells, and can be used for producing stimulated cell sheets that might be further implanted as a patch into a human body.

### BRIEF DESCRIPTION OF THE SEVERAL VIEWS OF THE DRAWING

The invention is clarified by drawings which do not completely cover and do not limit the scope of claims of the presented technical solution but simply illustrate an embodiment of the flexible device and its application for bio-cell in-vitro electrical and mechanical stimulation and characterization.

FIG. 1 A setup for patient customization of implants using the device described herein prior to implantation. Cells can be grown on device with geometry customized to eventual implantation sight. Electrical and mechanical stimulation profiles can be derived from specific patient or optimized data and sent to device while conduction velocity and contractility cell response can be monitored in real time. Stimulation profiles can be iterated upon until the patch meets performance requirements and can be released for implantation.

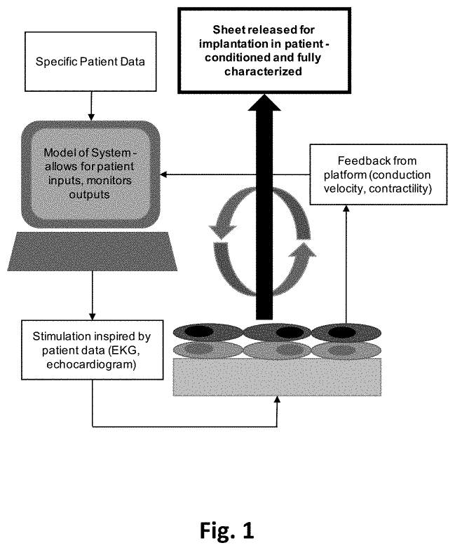

FIG. 2 Design of the device: cross-section of wall profile, groove/ridge architecture, cell locations and alignment of cells perpendicular to wall direction are shown. Top down view of the device shows orientation of cells in relationship to wall and groove/ridge directions. The benefit of the positive sloped groove/ridge profile during gold deposition is depicted; it allows for continuous gold deposition along the top of the wall. The benefit of the negative sloped side walls during gold deposition is depicted; this wall profile prevents electrical shorting between walls after deposition.

FIG. 3 Two configurations for electrical stimulation. In both of these configurations, the user is able to manipulate voltage, frequency, and pulse duration and shape. On the left, in the direct contact to contact stimulation, the user can directly stimulate individual cells, readout the current through the device, and analyze effects of conditioning on cells by measuring alignment, protein content, contractility, etc. of cells before and after stimulation. On the right, in the contact to common ground stimulation, the user can readout the time for signal to travel from one gold coated side of the device to the other, allowing for measurements of conduction velocity.

FIG. 4 Setup for mechanical motion detection. Cells are imaged with a long working distance objective from the underside of the device as seen in the top portion of the drawing. The displacement of the top of the wall caused by the cell contraction is used to calculate the force that they are applying. The microscope is focused on the underside of the top of the wall (which is coated in gold); since the gold is non-transparent, the light source from above effectively silhouettes the wall top surface, making the portions of the image that the user wants to track appear like parallel, black bars.

### DETAILED DESCRIPTION OF THE INVENTION

### Design

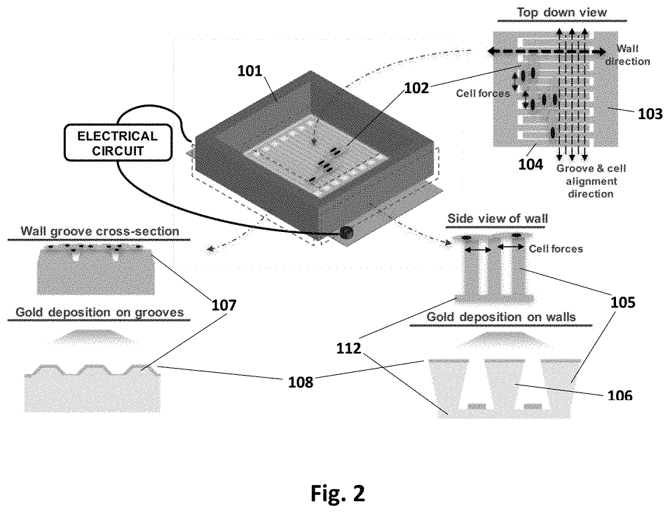

This device has been designed to achieve the following: cellular alignment, electrical stimulation, mechanical stimulation, conduction velocity readout, contraction force readout, and upon characterization, cell sheet release. In brief, the device achieves these design criteria through the following setup. The platform is based on a set of interdigitated comb electrical contacts 104. These are three-dimensional walls made of polydimethylsiloxane (PDMS) 105 coated with electrically conductive films of titanium and gold 108. Not only do the walls serve as a method for stimulating cells that are sitting on top of them, but their geometry and physical properties (namely stiffness) can be tailored to make them flexible (or rigid) enough to be bent by the range of forces applied by cells during contraction. In this way, they can be used to measure force throughout the cell culture, at the cellular or cell-group level. The walls 105 also play the role of a substrate that has an effective modulus more relevant to muscle cell culture than tissue culture plastic or planar PDMS. The entire platform is flexible and can be stretched via a linear actuator setup, allowing for simultaneous electrical and mechanical stimulation. Device details are shown in FIG. 2.

In order for the device to function, it is crucial that there be a method for predictably aligning the cells 102 perpendicular, or at a certain angle, to the walls/contacts of the device. This is to (1) mimic myocardial cellular architecture, (2) allow a current flowing from the positive comb finger contact to the negative comb finger contact to pass through the cell (effectively stimulating it), and (3) if cells do contract, they are able to move the wall for a mechanical readout. In order to align the cells, a groove/ridge architecture is used on the tops of all walls such that the cells orient themselves perpendicular, or at a certain angle, to the walls. In this design, the ridges are short and shallow in comparison to the wall height and are estimated to negligibly affect the motion of the walls. A 3 mm high PDMS square well 101 is attached to the device to isolate cells and media on the comb platform. Electrical connections to the side comb buses 103 are made outside of the well.

In order to tailor the geometry to have the correct mechanics to be compatible with cellular forces, theoretical calculations and Comsol simulations were run to determine the wall geometry necessary for bending. In particular, a Young's modulus of 750 kPa and Poisson's ratio of 0.49 were used (these are values for PDMS mixed at a 10:1 ratio and cured at 80° C. for 12 hours) for designing a specific device possessing certain mechanical performance. It is necessary that the shear force applied by cellular contraction move the wall enough that it's displacement is detectable by the microscope 111.

### Fabrication

In brief, the fabrication steps include preparing an auxiliary silicon mold using established photolithography techniques. One mask is used for pattering grooves followed by standard KOH etching; the second mask is used for patterning walls followed by Bosch deep reactive ion etching and finally cleaning of the mold. The polymer PDMS is cast into the mold to create the three-dimensional grooved interdigitated comb-wall architecture, coating the top of this device with conductive (standard titanium/gold) film, and finally attaching a well for cell isolation and wires for electrical stimulation and readout as seen in FIG. 2.

### Cellular Survival and Alignment on Device

Different combinations of groove, ridge and wall width result in varying degrees of cellular alignment. Alignment can be achieved with the correct ratio of groove width to overall wall width; this must be tailored to the cell source. Stimulation seems to enhance cellular alignment in some cases. On devices with poor alignment (cells align parallel to walls), narrower wall widths cause the cells to align with the walls and not perpendicular to them. Larger gaps allow the cells to crawl down into them, preventing cells from touching two contact fingers and from forming sheets on top of the device. Larger groove ridge patterns are too wide and the cells failed to consistently recognize the architecture.

### Electrical Stimulation and Conduction Velocity Measurement

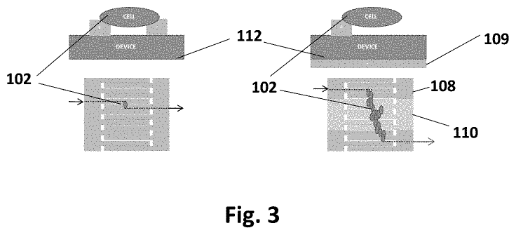

There are two modes of stimulation as shown in FIG. 3: direct contact 104-to-contact 104 shown on the left and contact 104-to-common ground 109 shown on the right. In direct contact-to-contact, an electrical pulse generated by a function generator is applied to the sets of comb electrical fingers 104. The signal wire is connected to one side contact bus 103 while the second comb fingers are grounded. A pulse voltage is applied to the cells contacting adjacent fingers. This mode is used for conditioning cells with input stimuli. In the second mode (contact to common ground), an electrical pulse is applied to only one set of fingers while the electrically isolated, copper ground plate 109 is placed under the device. The second set of fingers is used for detecting pulse propagation of the signal that has been passed by the cells across the non-conducting PDMS section in between. This mode is used for measuring conduction velocity. Devices are stimulated at one gold band and signals that travel across the non-conducting PDMS only sections to the next gold band are visualized on an oscilloscope; the time is measured in between the input signal pulse and the detected signal pulse. The length of the PDMS only gap between the gold conductive sections is measured for calculating the conduction velocity as a ratio of the gap over time between input and detected pulses.

### Detection of Mechanical Motion

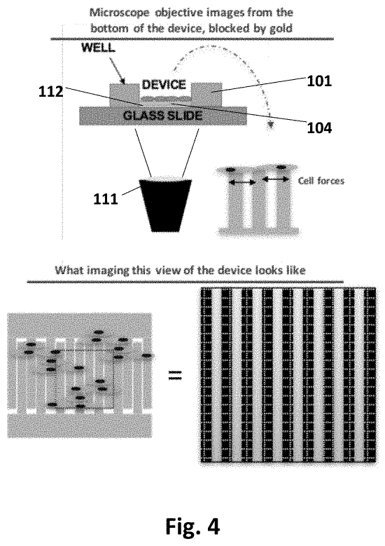

Devices with cells seeded can be visualized using an Olympus 1×81 microscope with an environmental chamber set to 37° C., 5% CO2. A 20×, long working distance objective 111 is used to focus on the underside of the wall top surface of the device as shown in FIG. 4. The non-transparent gold coating is leveraged for imaging. Viewing from underneath the device, wall motion can be tracked, as gold-coated wall tops show up as parallel black bars with easily tracked movement, and irrelevant cellular body motion is reduced in the recorded image. With the correct contrast and using a custom computer vision code built using Python openCV code to detect motion, the wall displacements can be tracked over time. For wall displacements detectable by the microscope, the aspect ratio of the walls (height/width) needs to be at least 3:1, given PDMS properties (stiffness).

### Simultaneous Linear Actuation and Electrical Stimulation

The bio-MEMS device described here has been designed to coordinate electrical stimulation according to electrocardiogram (EKG) signaling with linear motion approximated by left ventricular volume change during a beat cycle (as derived from echocardiogram ECHO). The coordination of the mechanical and electrical stimulation is realized by using one- or two-direction translational stages or any other mechanical drivers that provide reciprocating motion of the edge(s) of the PDMS base 112. By adding a trigger mechanism or circuit to the mechanical driver different phase shifts (lead/lag) in mechanical and electrical stimulation can be realized. Mechanical and electrical stimulation have to be synchronized, and a phase/time shift between them may be adjusted.

### Triggered Cell Sheet Release

Cell sheet release should be achievable by one of two published methods (1) attaching to gold surface a custom synthetic oligopeptide, CCRRGDWLC, according to a published protocol that allows cells to bind and then be released as a confluent sheet by applying −1V across the device (J. Enomoto et al, Regenerative Therapy, 2016, 3, 24-31) or (2) using the alginate hydrogel cell sheet technique described in U.S. patent Ser. No. 14/891,713 in combination with the device.

What is claimed is:

1. Conductive film coated device for one-directional alignment and electrical stimulation of seeded biological cells and control of resulting mechanical contraction and conduction velocity, comprising
a. a non-conductive substrate on which a grid of inter-digitated parallel walls is formed and each said wall features periodical hills and valleys patterned along the top of the wall and negative slope side walls, and
b. a conductive film on top of the walls, that is physically and electrically connected to the device side external common contacts.

2. Device of claim 1 comprising more than 1 inter-digitated pair of electrically isolated contact combs.

3. Device of claim 1 in which the angle between direction of the walls and direction of ridges/grooves is chosen between 45 and 90 degrees.

4. Device of claim 1 comprising a common ground electrode under it.

5. Device of claim 1 featuring the aspect ratio of the walls (height/width) between 3 and 12.

6. Device of claim 1 featuring the groove depth between 1 and 15 microns.

7. Device of claim 1 applied for electrical stimulation of iPS-derived cardiomyocytes.

8. Device of claim 1 applied for simultaneous electrical and mechanical stimulation of iPS-derived cardiomyocytes.

9. Device of claim 1 in which the interstitial space between walls is filled in with a soft gel.

10. Conductive film coated device for alignment and combined electrical and mechanical stimulation of seeded biological cells and control of resulting mechanical contraction and conduction velocity, comprising
a. a non-conductive substrate on which a grid of inter-digitated parallel walls is formed and each said wall features periodical hills and valleys patterned along the top of the wall and negative slope side walls, and
b. a conductive film on top of the walls, that is physically and electrically connected to the device side common contacts, and
c. one or two electrically controlled linear actuators providing reciprocating motion with a frequency preferably in a range between 0.5 Hz and 5 Hz, firmly attached to one or two perpendicular sides of the substrate while other three or other two sides of the substrate are firmly attached to a heavy stand and fixed.

11. Device of claim 10 comprising more than 1 inter-digitated pair of electrically isolated contact combs.

12. Device of claim 10 in which the angle between direction of the walls and direction of ridges/grooves is chosen between 45 and 90 degrees.

13. Device of claim 10 comprising a common ground electrode under it.

14. Device of claim 10 featuring the aspect ratio of the walls (height/width) between 0.5 and 12.

15. Device of claim 10 featuring the groove depth between 1 and 15 microns.

16. Device of claim 10 applied for electrical stimulation of iPS-derived cardiomyocytes.

17. Device of claim 10 applied for simultaneous electrical and mechanical stimulation of iPS-derived cardiomyocytes.

18. Device of claim 10 in which the interstitial space between walls is filled in with a soft gel.
